(19) United States
(12) Patent Application Publication
(10) Pub. No.: US 2008/0025931 A1
(43) Pub. Date: Jan. 31, 2008

(54) Cosmetic method for limiting age-related hollowing of the face

(76) Inventors: Pascale Pelletier, Antony (FR); Catherine Marion, Antony (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/822,162

(22) Filed: Jul. 3, 2007

Related U.S. Application Data
(60) Provisional application No. 60/830,113, filed on Jul. 12, 2006.

(30) Foreign Application Priority Data
Jul. 3, 2006 (FR) 0652782

(51) Int. Cl. A61K 8/99; A61K 8/96; A61Q 17/04; A61Q 19/00

(52) U.S. Cl. 424/59; 424/195.16; 424/780

(57) ABSTRACT

Disclosed herein is a cosmetic method for remodelling the face and/or limiting age-related hollowing of the face, by applying to the skin of the face and/or the neck, a composition containing, in a physiologically acceptable medium, at least one extract of a non-fruiting non-photosynthetic filamentous bacterium.

This application claims benefit of U.S. Provisional Application No. 60/830,113, filed Jul. 12, 2006, the contents of which are incorporated herein by reference. This application also claims benefit of priority under 35 U.S.C. § 119 to French Patent Application No. FR 06 52782, filed Jul. 3, 2006, the contents of which are also incorporated herein by reference.

Disclosed herein is a cosmetic method for reducing the melting of the lipostructure of the skin in the face and/or the neck, and/or for avoiding the sagging and/or hollowing of the volumes of the face, by applying to the skin of the face and/or the neck a composition containing, in a physiologically acceptable medium, at least one extract of a non-fruiting non-photosynthetic filamentous bacterium.

Further disclosed herein is a cosmetic composition which can be used in the method according to the present disclosure, containing, in a physiologically acceptable medium, (i) at least one extract of a non-fruiting non-photosynthetic filamentous bacterium and (ii) at least one C-glycoside derivative.

The present disclosure further relates to the cosmetic use of at least one extract of a non-fruiting non-photosynthetic filamentous bacterium in a composition, to reduce the loss of skin density and/or the melting of the lipostructure of the skin and/or avoid the sagging and/or hollowing of the volumes of the facet.

It is known that the signs of skin aging have chronological causes (genetically programmed aging) and additional aggravating factors such as exposure to UV radiation and hormone deficiencies occurring during menopause. The result typically is changes in the state of the skin that may be due to a slowing down of and a disequilibrium in skin function, resulting in “atrophy” of all the strata of the skin. A decrease in the quality of the dermis (elastin, collagen, glycosaminoglycans) and a loss of consistency of the extracellular matrix; a decrease in the thickness of the epidermis (slowing down of cell renewal); a slowing down of the production of lipids and of the proteins of the epidermal structure; a disequilibrium of desquamation; and/or a reduction in the water content of the skin, may be observed.

For example, following the abovementioned changes, it may be observed that the supporting mattress of the skin is weakened and the adipose mass melts. This is also referred to as melting of the “lipostructure” of the skin.

The expression “lipostructure of the skin” as used herein is understood to mean the network of lipid cells which forms the volumes on which the facial skin rests and is molded.

The face may thus gradually hollow, the skin may lose its consistency and its support, the skin may fall; the oval may become heavy; and sagging and/or hollowing of the volumes of the face may be observed.

This phenomena is especially visible on the cheeks, which become increasingly hollow, on the contour of the eye and on the oval of the face, which is less well defined (appearance of jowls).

These phenomena may become more pronounced with age and may be particularly noticeable in people with mature or even very mature skin, and for example in menopausal women.

The expression “mature skin” as used herein is understood to mean skin of subjects who are at least 40 years old.

The expression “very mature skin” as used herein is understood to mean skin of subjects who are at least 50 years old, for example, at least 60 years old, or even 65 years old.

Means are being constantly sought for combating, or at least for delaying, the appearance of these signs of aging, and new means are being sought which make it possible to:
reduce the loss of skin density and/or the melting of the lipostructure of the skin, for example in the cheeks and the contour of the eye, and/or
restore the lipostructure of the skin, and/or
avoid the sagging and/or hollowing of the volumes of the face, for example in the cheeks and the contour of the eye, and/or
increase the volume of the cheekbones, and/or
improve the volumes underlying the skin of the face and/or of the neck, for example in the cheeks, the oval of the face and the contour of the eye, and/or
improve the skin density, plumpness and support, for example in the cheeks, the oval of the face and the contour of the eye, and/or
reshape the facial features, for example the oval of the face.

The present inventors have discovered, surprisingly and unexpectedly, that an extract of a non-fruiting non-photosynthetic filamentous bacterium has the property of stimulating lipogenesis and promoting adipocyte differentiation, thus making it possible to avoid or slow down the melting of the fat contained in the supporting tissues of the skin, otherwise called “melting of the lipostructure of the skin”.

Disclosed herein, therefore, is the use of such an extract of a non-fruiting non-photosynthetic filamentous bacterium for combating one or more of the various mechanisms of hollowing and sagging of the face and/or the neck.

Extracts of a non-fruiting non-photosynthetic filamentous bacterium are already known as immunostimulating agents (EP 0 604 631), substance P antagonists (EP 0 761 204), agents stimulating the proliferation of the fibroblasts (EP 0 681 831), antioxidants (EP 1 354 593), or alternatively slimming agents (FR 99 00405, now issued as FR 2 788 434)

However, to the knowledge of the present inventors, the effect of these extracts of a non-fruiting non-photosynthetic filamentous bacterium, for example, Vitreoscilla filiformis, on the stimulation of lipogenesis and/or the differentiation of the adipocytes, or their use for remodelling the face and/or the neck and/or limiting age-related hollowing of the face, is not known in the art.

Accordingly, disclosed herein is a cosmetic method for reducing the melting of the lipostructure of the skin in the face and/or the neck, and/or for avoiding the sagging and/or hollowing of the volumes of the face, by applying to the skin of the face and/or the neck a composition comprising, in a physiologically acceptable medium, at least one extract of a non-fruiting non-photosynthetic filamentous bacterium.

Also disclosed herein is the use of the composition, as described above, to increase the volume of the cheekbones.

The disclosure further relates to the use of the composition, as described above, to redefine the contours and/or the oval of the face, and/or to avoid the formation of jowls and/or to limit the hollowing of the cheeks and/or of the contour of the eye.

According to at least one embodiment of the present disclosure, the composition is applied to the areas of age-related hollowing and/or sagging of the face and/or the neck or in such areas of the face and/or neck to people with hollowing of the cheeks and/or the contour of the eye and/or sagging of the cheeks.

These areas of age-related hollowing and/or sagging may be, for example, the cheeks, the contour of the eye and/or the oval of the face (contours of the face).

People with hollowing of the cheeks and/or the contour of the eye and/or sagging of the cheeks may be men and women of any age, for example, people with mature skin (at least 40 years old), or very mature skin (at least 50 or even 60 years old), or menopausal women.

The method disclosed herein therefore may be advantageous, for example, for remodelling the face and/or the neck and/or for limiting the hollowing of the face of people with mature or even very mature skin.

It also may be advantageous for remodelling the face and/or the neck and/or for limiting the hollowing of the face of menopausal women.

Extracts of a Non-Fruiting Non-Photosynthetic Filamentous Bacterium

The extracts of bacteria which can be used according to the present disclosure are prepared from non-photosynthetic filamentous bacteria as defined according to the classification of Bergey's Manual of Systematic Bacteriology (vol. 3, sections 22 and 23, 9th edition, 1989), such as, for example, bacteria belonging to the order Beggiatoales, and bacteria belonging to the genera Beggiatoa, Vitreoscilla, Flexithrix or Leucothrix.

The bacteria which have just been defined and several of which have already been described generally have an aquatic habitat and may be found in particular in sea water or in thermal water. For example, the following bacteria may be used:

Vitreoscilla filiformis (ATCC 15551)

Vitreoscilla beggiatoïdes (ATCC 43181)

Beggiatoa alba (ATCC 33555)

Flexithrix dorotheae (ATCC 23163)

Leucothrix mucor (ATCC 25107)

Sphaerotilus natans (ATCC 13338)

According to at least one embodiment, an extract of Vitreoscilla filiformis (ATCC 15551) may be used.

The expression “bacterial extract” as used herein is understood to mean an extract of the bacterial biomass or any active fraction of the extract, for example:
(i) bacterial cells isolated from the culture medium, which have been concentrated, for example by centrifugation (“non-stabilized cell extract”); or
(ii) bacterial cells concentrated (i), and then subjected to an operation of breaking the envelopes of the bacterial cells by any means known to persons skilled in the art, such as the action of ultrasound or, for instance, autoclaving (“stabilized cell extract”). The expression “envelopes” as used herein is understood to mean bacterial wall and optionally the subjacent membranes;
(iii) the supernatant obtained by filtration of the stabilized cell extract (ii), or any active fraction of the extract having a stimulating effect on lipogenesis and/or adipocyte differentiation.

This active fraction, whose effect on lipogenesis and/or adipocyte differentiation may be evaluated according to one of the methods described in the examples below, may be obtained by conventional fractionation methods such as extraction in the presence of a solvent, selective precipitation or tangential ultrafiltration (TUF) for example.

These extracts or fractions may be preserved, for example, by freezing the extracts or the fractions and used after thawing.

The expressions “cell extract” of bacteria ((i) and (ii)), “supernatant” of the said extract (iii) or “active fraction” will be more simply used in the remainder of the description.

The extract of a non-fruiting non-photosynthetic filamentous bacterium which can be used in the composition used in the method disclosed herein may be chosen from a cell extract, the supernatant of the cell extract or an active fraction of the cell extract.

According to at least one embodiment, the extract of a non-fruiting non-photosynthetic filamentous bacterium is an extract of Vitreoscilla filiformis, such as a cell extract of Vitreoscilla filiformis.

To prepare the bacterial extract according to the present disclosure, the bacteria may be cultured according to methods known to persons skilled in the art, or reference may be made, for example, to the description of international patent application WO-A-94-02158. A cell extract is obtained whose supernatant may be separated for example by filtration and centrifugation. The extract may be used in aqueous form or in freeze-dried form. The protocol is described in greater detail in Example 1 below.

This bacterial extract may be refractionated and used pure or diluted at various concentrations.

According to at least one embodiment, from 0.001 to 10% by weight, such as from 0.005 to 2% by weight, of dry extract of a non-fruiting non-photosynthetic filamentous bacterium relative to the total weight of the composition, may be used.

According to yet another embodiment, from 0.01 to 2% by weight of dry extract of a non-fruiting non-photosynthetic filamentous bacterium, relative to the total weight of the composition may be used.

These compositions may contain the extract of a non-fruiting non-photosynthetic filamentous bacterium in the form of a dispersion in an appropriate vehicle such as, for example, water, organic solvents, fatty substances including oils, and mixtures thereof, for example, emulsions.

The composition used in the method disclosed herein is generally suitable for topical application to the skin and therefore may comprise a physiologically acceptable medium, for example, a medium compatible with the skin and/or its superficial body growths. Such a cosmetically acceptable medium, may be, for example, a medium which has a pleasant color, odor or feel and does not cause unacceptable discomfort (prickling, tightness, blotches) capable of putting the consumer off from using this composition.

The composition disclosed herein may be provided in any of the galenic forms conventionally used for topical application, for example, in the form of dispersions of the lotion or aqueous gel type, of emulsions with a liquid or semiliquid consistency of the milk type, obtained by dispersing a fatty phase in an aqueous phase (O/W) or conversely (W/O), or of suspensions or emulsions with a soft, semisolid or solid consistency of the cream, gel or serum type, or alternatively of multiple emulsions (W/O/W or O/W/O), of microemulsions, of vesicular dispersions of the ionic and/or non-ionic type, or of wax/aqueous phase dispersions. These compositions are prepared according to the customary methods.

According to one embodiment of the invention, the composition is provided in the form of an oil-in-water (O/W) emulsion.

This will be, for example, a fluid, a cream, a gel or a serum, for example a day cream, a night cream, a serum for the contour of the eyes.

Oils which may be used in the composition include, for example: fatty acid esters of fatty alcohols; silicone oils which are volatile (such as cyclomethicones) or non-volatile (such as dimethicones); branched fatty alcohols such as octyldodecanol; hydrocarbon oils such as petroleum jelly, squalane, isohexadecane and mineral oils; vegetable oils; and shea butter. The oily phase may also comprise waxes such as beeswax.

The composition may additionally contain various adjuvants commonly used in the cosmetic field, such as polyols, such as glycerine, propylene glycol and polyethylene glycols; emulsifiers such as fatty acid esters of polyethylene glycol, fatty acid esters of glyceryl, fatty acid esters of sucrose, fatty alcohol ethers of polyethylene glycol, and optionally oxyethylenated fatty acid esters of methylglucose; coemulsifiers such as cetyl and stearyl alcohols; fillers, such as silica, and expanded powders such as the microspheres formed of a terpolymer of vinylidene chloride, acrylonitrile and methacrylate and marketed under the name EXPANCEL by the company Kemanord Plast; thickeners and/or gelling agents such as polysaccharide or silicone gums, homo- and copolymers of acrylamide, homo- and copolymers of acrylic acid and homo- and copolymers of acrylamidomethylpropanesulphonic acid (AMPS); preservatives; sequestrants; pH regulators such as triethanolamine, sodium hydroxide or citric acid; ethanol; colorants; pearlescent agents; and perfumes.

Of course, persons skilled in the art will be careful to choose from these optional additional compounds and their quantities such that the advantageous properties of the composition disclosed herein is not substantially impaired by the addition envisaged.

The composition used in the method disclosed herein makes it possible to slow down the loss of material from the face which is responsible for its hollowing and for its sagging with age. To reinforce the effects of this composition, the latter may additionally contain at least one agent chosen from: agents promoting the synthesis of dermal and/or epidermal macromolecules, agents stimulating the proliferation of keratinocytes, agents promoting the synthesis of epidermal lipids, agents stimulating cell metabolism and agents promoting microcirculation.

Agents Promoting the Synthesis of Dermal or Epidermal Macromolecules and/or Preventing their Degradation

Agents increasing the synthesis of glycosaminoglycans include, for example: a product of fermentation of milk by Lactobacillus vulgaris, such as that marketed by the company BROOKS under the trade name Biomin yogourth®; an extract of brown alga Padina pavonica such as that marketed by the company ALBAN MÜLLER under the trade name HSP3®; a Saccharomyces cerevisiae extract available in particular from the company SILAB under the trade name Firmalift® or from the company LSN under the trade name Cytovitin®; a Laminaria ochroleuca extract such as that available from the company SECMA under the trade name Laminaine®; a Centella asiatica extract such as that available from the company ROCHE under the trade name ETCA®; a watercress (Nasturtium officinale) extract available from the company SILAB under the trade name Odraline®; Mamaku essence from Lucas Meyer; D-xylose, its esters and oligosaccharides containing D-xylose as described, for example, in international application WO 99/24009; the C-glycosides described in international application WO 02/051828, such as C-β-D-xylopyranoside-2-hydroxy-propane and its salts and optical and geometric isomers. In at least one embodiment, an agent that promotes the synthesis of glycosaminoglycans is C-β-D-xylopyranoside-2-hydroxy-propane and its salts and optical and geometric isomers.

Agents promoting the synthesis of collagen and/or prevent its degradation include, for example: Centella asiatica extracts, asiaticosides and derivatives; ascorbic acid or vitamin C and its derivatives; synthetic peptides such as iamin, biopeptide CL or palmitoyloligopeptide marketed by the company SEDERMA; peptides extracted from plants, such as the soybean hydrolysate marketed by the company COLETICA under the trade name Phytokine® or the hydrolysed soybean protein marketed by the company SILAB under the name Ridulisse® and plant hormones such as auxins, and lignans; retinoids and derivatives, malt extract marketed by the company COLETICA under the trade name Collalift®; bilberry or rosemary extracts; lycopene. In at least one embodiment, an agent promoting the synthesis of collagen is a hydrolyzed soybean protein marketed by the company SILAB under the name Ridulisse®.

Agents promoting the synthesis of epidermal molecules (e.g.: fillagrin and keratins), include, for example, the lupin extract marketed by the company SILAB under the trade name Structurine® and the beech Fagus sylvatica bud extract marketed by the company GATTEFOSSE under the trade name Gatuline® RC.

Agents Promoting the Proliferation of Keratinocytes

Agents promoting the proliferation of keratinocytes include, for example: phloroglucinol; nut oil cake extracts marketed by the company GATTEFOSSE; Solanum tuberosum extracts marketed by the company SEDERMA; a Larrea divaricata extract such as Capislow® from Sederma, mixtures of papaya, olive and lemon leaves such as Xyleine from Vincience, Hydrangea macrophylla leaf extract such as Amacha liquid EE from Ichimaru Pharcos; and a yeast extract such as Stimoderm® from CLR.

Agents Promoting the Synthesis of Epidermal Lipids

Agents promoting the synthesis of epidermal lipids include, for example, ascorbic acid and its derivatives, and cinnamic acid and its derivatives.

Agents Stimulating Cell Metabolism

Agents stimulating the energy metabolism of cells, which is slowed down during aging, include, for example: biotin; a Saccharomyces cerevisaie extract such as Phosphovital® from Sederma; Physiogenyl® from Solabia; and a mixture of zinc, copper and magnesium gluconate such as Sepitonic M3® from Seppic.

Agents Promoting Skin Microcirculation

Agents promoting skin microcirculation include, for example: caffeine, extracts of ruscus, horse chestnut, ivy, ginseng, melilot, KOMBUCHKA from Sederma, pycnogenol, manganese gluconate (GIVOBIO GMn from Seppic), VISNADIN from Indena, a lupin extract (ECLALINE from Silab), EPALINE 100 from Laboratoires Carilbne, a Seville orange flower extract (REMODULINE from ilab), vitamin P and its derivatives such as PERMETHOL from Sochibios, nicotinate and derivatives, lysine and derivatives (such as ASPARLYNE from Solabia). In at least one embodiment the agent that promotes skin microcirculation is caffeine, ruscus extract, or horse chestnut extract.

Agents promoting skin microcirculation may be used in compositions intended for the contour of the eyes.

These additional agents will be present in the composition in amounts ranging from 0.001 to 10% by weight relative to the total weight of the composition. For example, these additional agents will be present in the composition in amounts ranging from 0.01 to 1% by weight relative to the total weight of the composition.

For example, the composition used in the method according to the present disclosure comprises (i) at least one extract of a non-fruiting non-photosynthetic filamentous bacterium and (ii) at least one agent increasing the synthesis of glycosaminoglycans as agent promoting the synthesis of dermal and/or epidermal macromolecules.

The composition may additionally comprise a combination of three active agents comprising an agent increasing the synthesis of collagen as agent promoting the synthesis of dermal and/or epidermal macromolecules, an agent increasing the synthesis of epidermal lipids, and an agent promoting the proliferation of keratinocytes.

According to at least one embodiment, in addition to this combination of three active agents, the composition may comprise at least one agent stimulating cell metabolism and/or at least one agent promoting microcirculation.

The agent stimulating cell metabolism may be used, for example, for night compositions and the agent promoting microcirculation may be used, for example, for compositions intended for the contour of the eye.

For example, in at least one embodiment:
the extract of a non-fruiting non-photosynthetic filamentous bacterium is a Vitreoscilla filiformis extract;
the agent increasing the synthesis of glycosaminoglycans is a C-glycoside derivative as described in international application WO 02/051828, such as C-β-D-xylopyranoside-2-hydroxy-propane or one of its salts or optical and geometric isomers;
the agent promoting collagen synthesis is a hydrolysed soybean protein such as that marketed by SILAB under the name RIDULISSE®;
the agent promoting the synthesis of epidermal macromolecules is a beech Fagus sylvatica bud extract such as that marketed by the company GATTEFOSSE under the trade name Gatuline® RC;
the agent increasing the proliferation of keratinocytes is phloroglucinol (1,3,5-tri-methoxybenzene) or one of its derivatives;
the agent promoting the synthesis of epidermal lipids is cinnamic acid or one of its derivatives;
the agent stimulating cell metabolism is a Saccharomyces cerevisiae extract such as Phosphovital® from Sederma;
the agent promoting skin microcirculation is caffeine.

The composition according to the present disclosure may additionally contain at least one UVA and/or UVB screening agent. The sunscreens may be chosen from organic screening agents, inorganic screening agents and mixtures thereof.

For example, the organic screening agents may be chosen from the following (cited according to the CTFA nomenclature): Ethylhexyl Salicylate, Homosalate, Ethylhexyl Methoxycinnamate, Butyl Methoxydibenzoylmethane, Octocrylene, Phenylbenzimidazole Sulfonic Acid, Disodium Phenyl Dibenzimidazole Tetra-sulfonate, Benzophenone-3, Benzophenone-4, Benzophenone-5,4-Methylbenzylidene camphor, Terephthalylidene Dicamphor Sulfonic Acid, Bis-Ethylhexyloxyphenol Methoxyphenyl Triazine, Ethylhexyl triazone, Diethylhexyl Butamido Triazone, Methylene bis-Benzotriazolyl Tetramethylbutylphenol, Drometrizole Trisiloxane, Polysilicone-15, and from the following (cited as chemical names): n-hexyl 2-(4-diethylamino-2-hydroxybenzoyl)-benzoate, 1,1-dicarboxy-(2,2′-dimethyl-propyl)-4,4-diphenylbutadiene, 2,4-bis-[5-1 (dimethylpropyl)benzoxazol-2-yl-(4-phenyl)-imino]-6-(2-ethylhexyl)-imino-1,3,5-triazine, and mixtures thereof.

For example, the inorganic screening agents may be chosen from pigments or alternatively nanopigments (mean size of the primary particles: generally ranging from 5 nm to 100 nm, such as from 10 nm to 50 nm) of coated or uncoated metal oxides such as for example nanopigments of titanium oxide (amorphous or crystallized in rutile and/or anatase form), of iron, zinc, zirconium or cerium oxide.

Also disclosed herein is a cosmetic composition which can be used in the method disclosed above, containing, in a physiologically acceptable medium, (i) at least one extract of a non-fruiting non-photosynthetic filamentous bacterium and (ii) at least one C-glycoside derivative.

In at least one embodiment of the present disclosure, the extract of a non-fruiting non-photosynthetic filamentous bacterium is a Vitreoscilla filiformis extract. In another embodiment, the extract of a non-fruiting non-photosynthetic filamentous bacterium is a Vitreoscilla filiformis cell extract.

The C-glycoside derivatives which can be used in the composition as disclosed herein are described in international application WO 02/051828.

A suitable C-glycoside derivative for use herein may be a compound of the following formula (I): 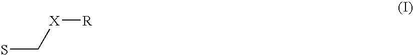
wherein:
R is chosen from:
   saturated C₁ to C₂₀, such as C₁ to C₁₀, and unsaturated C₂ to C₂₀, such as C₂ to C₁₀, linear alkyl radicals, and saturated or unsaturated C₃ to C₂₀, such as C₃ to C₁₀, branched or cyclic alkyl radicals;
   saturated C₁ to C₂₀, such as C₁ to C₁₀, and unsaturated C₂ to C₂₀, such as C₂ to C₁₀, linear, and saturated or unsaturated C₃ to C₂₀, such as C₃ to C₁₀, branched or cyclic, hydrofluoro- or perfluoroalkyl radicals;

wherein the hydrocarbon chain constituting the radicals may be interrupted by 1, 2, 3 or more heteroatoms chosen from:
oxygen,
sulphur,
nitrogen, and
silicon,

and be optionally substituted with at least one radical chosen from:
—OR₄,
—SR₄,
—NR₄R₅,
—COOR₄,
—CONHR₄,
—CN,
halogen atoms,
C₁ to C₆ hydrofluoro- or perfluoroalkyl radicals, and
C₃ to C₈ cycloalkyl radicals,

wherein R₄ and R₅ may be chosen from, independently of each other, hydrogen atoms, saturated C₁ to C₃₀, such as C₁ to C₁₂, or unsaturated C₂ to C₃₀, such as C₂ to C₁₂, linear alkyl, perfluoroalkyl or hydrofluoroalkyl radicals, and from saturated or unsaturated C₃ to C₃₀, such as C₃ to C₁₂, branched or cyclic alkyl, perfluoroalkyl or hydrofluoroalkyl radicals; and C₆ to C₁₀ aryl radicals,
X is a radical chosen from the following groups:

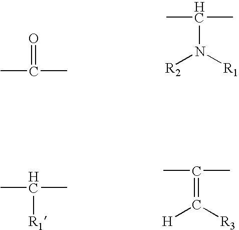

wherein R₁, R₂ and R₃ are chosen from, independently of each other, hydrogen and a radical R, as defined above, and R′₁ is chosen from a hydrogen atom, an —OH group, and a radical R as defined above, and wherein R₁ may also be chosen from C₆ to C₁₀ aryl radicals; and
S is chosen from monosaccharides and polysaccharides containing up to 20 sugar units, for example, up to 6 sugar units, in pyranose and/or furanose form and of the L and/or D series, wherein the mono- or polysaccharide may be substituted with a free hydroxyl group, and optionally at least one amine functional group which is optionally protected, and
the S—CH₂—X bond is a bond of a C-anomeric nature, which may be α or β; and its cosmetically acceptable salts, solvates such as hydrates and its isomers.

As disclosed herein, the expression “halogen” is understood to mean chlorine, fluorine, bromine or iodine.

The term “aryl” is understood to mean an aromatic ring such as phenyl, optionally substituted with one or more C₁-C₄ alkyl radicals.

The term “C₃ to C₈ cycloalkyl” is understood to mean an aliphatic ring having from 3 to 8 carbon atoms, including for example cyclopropyl, cyclopentyl and cyclohexyl.

Suitable alkyl groups may be chosen from, for example: methyl, ethyl, isopropyl, n-propyl, n-butyl, t-butyl, isobutyl, sec-butyl, pentyl, n-hexyl, cyclopropyl, cyclopentyl, cyclohexyl, and allyl groups.

According to at least one embodiment of the invention, it is possible to use a C-glycoside derivative corresponding to the formula (I) for which S may be chosen from monosaccharides and polysaccharides containing up to 6 sugar units, in pyranose and/or furanose form and of the L and/or D series, the mono- or polysaccharide having at least one free hydroxyl functional group and/or optionally at least one protected amine functional group, X and R moreover retaining all the definitions given above.

Monosaccharides useful herein may be chosen from D-glucose, D-galactose, D-mannose, D-xylose, D-lyxose, L-fucose, L-arabinose, L-rhamnose, D-glucuronic acid, D-galacturonic acid, D-iduronic acid, N-acetyl-D-glucosamine, N-acetyl-D-galactosamine and advantageously denotes D-glucose, D-xylose, N-acetyl-D-glucosamine or L-fucose, and in at least one embodiment, D-xylose.

Polysaccharides useful herein containing up to 6 sugar units may be chosen from D-maltose, D-lactose, D-cellobiose, D-maltotriose, a disaccharide combining a uronic acid chosen from D-iduronic acid or D-glucuronic acid with a hexosamine chosen from D-galactosamine, D-glucosamine, N-acetyl-D-galactosamine, N-acetyl-D-glucosamine, an oligosaccharide containing at least one xylose which may be chosen from xylobiose, methyl-β-xylobioside, xylotriose, xylotetraose, xylopentaose and xylohexaose and, for example, xylobiose which is composed of two xylose molecules linked by a 1-4 linkage.

In at least one embodiment, S is a monosaccharide chosen from D-glucose, D-xylose, L-fucose, D-galactose, D-maltose, such as D-xylose.

According to another embodiment of the present disclosure, it is possible to use C-glycoside derivatives corresponding to the formula (I) for which X is chosen from —CO—, —CH(OH)—, —CH(NR₁R₂)—, and —CH(R)—, for example chosen from, —CO—, —CH(OH)—, —CH(NH₂)—, —CH(NHCH₂CH₂CH₂OH)—, —CH(NHPh)-, and —CH(CH₃)—, such as from —CO—, —CH(OH)—, and —CH(NH₂)—, and in at least one embodiment, X is —CH(OH)—. For all of these embodiments, S and R retain all the definitions given above.

According to another embodiment of the invention, it is possible to use a C-glycoside derivative corresponding to the formula (I) for which R is chosen from saturated C₁ to C₂₀, such as C₁ to C₁₀, and unsaturated C₂ to C₂₀, such as C₂ to C₁₀, linear alkyl radicals, and saturated or unsaturated, C₃ to C₂₀, such as C₃ to C₁₀, branched or cyclic alkyl radicals, and optionally substituted as described above, S and R moreover retaining all the definitions given above. For example, R is chosen from linear C₁-C₄, such as C₁-C₃, radicals optionally substituted with —OH, —COOH or —COOR″₂, R″₂ being a saturated C₁-C₄ alkyl, for example, ethyl, radical. In at least one embodiment, R is an unsubstituted C₁-C₄, such as C₁-C₂, linear alkyl radical, such as ethyl.

Among C-glycoside derivatives of formula (I) that may be used herein, in at least one embodiment:
R is chosen from saturated C₁ to C₂₀, such as C₁ to C₁₀, and unsaturated C₂ to C₂₀, such as C₂ to C₁₀, linear alkyl radicals, and saturated or unsaturated C₃ to C₂₀ such as C₃ to C₁₀, branched or cyclic alkyl radicals, and optionally substituted as described above;
S is a monosaccharide as described above;
X is chosen from —CO—, —CH(OH)—, —CH(NR₁R₂)—, —CH(R)— as described above.

In at least one embodiment, a C-glycoside derivative of formula (I) is used for which:
R is chosen from C₁-C₄, such as C₁-C₃, linear radicals optionally substituted with —OH, —COOH or —COOR″₂, R″₂ being a saturated C₁-C₄ alkyl, for example, ethyl, radical;
S is a monosaccharide as described above;
X is chosen from —CO—, —CH(OH)—, —CH(NH₂)—, —CH(NHCH₂CH₂CH₂OH)—, —CH(NHPh)-, —CH(CH₃)—, for example, —CO—, —CH(OH)—, —CH(NH₂)—, and in at least one embodiment, X is —CH(OH)—.

In at least one embodiment, a C-glycoside derivative of formula (I) is used wherein:
R is chosen from unsubstituted C₁-C₄, such as C₁-C₂, linear alkyl, for example ethyl, radicals;
S is a monosaccharide as described above; for example, D-glucose, D-xylose, N-acetyl-D-glucosamine or L-fucose, and, in at least one embodiment, D-xylose;
X is chosen from —CO—, —CH(OH)—, —CH(NH₂)—, and in at least one embodiment, X is —CH(OH)—.

The acceptable salts for non-therapeutic use of the compositions described herein comprise conventional non-toxic salts of the compounds such as those formed from organic or inorganic acids. By way of example, there may be mentioned the salts of inorganic acids, such as sulphuric acid, hydrochloric acid, hydrobromic acid, hydriodic acid, phosphoric acid, boric acid. There may also be mentioned the salts of organic acids, which may comprise one or more carboxylic, sulphonic or phosphonic acid groups. They may be linear, branched or cyclic aliphatic acids or alternatively aromatic acids. These acids may additionally comprise at least one heteroatom chosen from O and N, for example in the form of hydroxyl groups. There may also be mentioned propionic acid, acetic acid, terephthalic acid, citric acid and tartaric acid.

When the compound of formula (I) comprises an acid group, neutralization of the acid group(s) may be carried out with an inorganic base, such as LiOH, NaOH, KOH, Ca(OH)₂, NH₄OH, Mg(OH)₂ or Zn(OH)₂; or with an organic base such as a primary, secondary or tertiary alkylamine, for example triethylamine or butylamine. This primary, secondary or tertiary alkylamine may comprise at least one atom chosen from nitrogen and oxygen atoms and may therefore comprise, for example, at least one alcohol functional group; there may be mentioned, for example, 2-amino-2-methylpropanol, triethanolamine, 2-dimethylaminopropanol, and 2-amino-2-(hydroxymethyl)-1,3-propanediol. There may also be mentioned lysine or 3-(dimethylamino)propylamine.

The acceptable solvates for the compounds described herein comprise conventional solvates such as those formed during the last step of preparation of the compounds because of the presence of solvents. By way of example, there may be mentioned the solvates due to the presence of water or of linear or branched alcohols such as ethanol or isopropanol.

By way of non-limiting illustration, examples of suitable C-glycoside derivatives include:
C-β-D-xylopyranoside-n-propane-2-one,
C-α-D-xylopyranoside-n-propane-2-one,
C-β-D-xylopyranoside-2-hydroxy-propane,
C-α-D-xylopyranoside-2-hydroxy-propane,
1-(C-β-D-fucopyranoside)-propane-2-one,
1-(C-α-D-fucopyranoside)-propane-2-one,
1-(C-β-L-fucopyranoside)-propane-2-one,
1-(C-α-L-fucopyranoside)-propane-2-one,
1-(C-β-D-fucopyranoside)-2-hydroxy-propane,
1-(C-α-D-fucopyranoside)-2-hydroxy-propane,
1-(C-β-L-fucopyranoside)-2-hydroxy-propane,
1-(C-α-L-fucopyranoside)-2-hydroxy-propane,
1-(C-β-D-glucopyranosyl)-2-hydroxyl-propane,
1-(C-α-D-glucopyranosyl)-2-hydroxyl-propane,
1-(C-β-D-galactopyranosyl)-2-hydroxyl-propane,
1-(C-α-D-galactopyranosyl)-2-hydroxyl-propane
1-(C-β-D-fucofuranosyl)-propane-2-one,
1-(C-α-D-fucofuranosyl)-propane-2-one
1-(C-β-L-fucofuranosyl)-propane-2-one,
1-(C-α-L-fucofuranosyl)-propane-2-one,
C-β-D-maltopyranoside-n-propane-2-one,
C-α-D-maltopyranoside-n-propane-2-one
C-β-D-maltopyranoside-2-hydroxy-propane,
C-α-D-maltopyranoside-2-hydroxy-propane, isomers thereof and mixtures thereof.

According to at least one embodiment, C-β-D-xylopyranoside-2-hydroxy-propane or C-α-D-xylopyranoside-2-hydroxy-propane, and C-β-D-xylo-pyranoside-2-hydroxy-propane, may be used for the preparation of a composition according to the present disclosure.

According to at least one embodiment, the C-glycoside derivative is C-α-D-xylopyranoside-2-hydroxy-propane in the form of a solution containing 30% by weight of active material in a water/propylene glycol mixture (60/40% by weight) such as the product manufactured by CHIMEX under the trade name “MEXORYL SBB®”.

As disclosed herein C-glycoside derivative corresponding to formula (I) may be used alone or as a mixture with other C-glycoside derivatives or in any proportion.

A C-glycoside derivative suitable for use herein may be obtained, in at least one embodiment, according to the method of synthesis described in the international application WO 02/051828.

The quantity of C-glycoside derivative to be used in a composition according to the present disclosure depends on the desired cosmetic or therapeutic effect, and may therefore vary widely.

Persons skilled in the art may, on the basis of their general knowledge, determine the appropriate quantities.

A composition according to the present disclosure may comprise a C-glycoside derivative present in an amount of 0.0001% to 25% by weight of active material relative to the total weight of the composition, for example, 0.001% to 10% by weight of active material, and in at least one embodiment, 0.05% to 5% by weight of active material of C-glycoside derivative relative to the total weight of the composition.

Further disclosed herein is a cosmetic composition which can be used in the method of the present disclosure, comprising, in a physiologically acceptable medium, (i) at least one extract of a non-fruiting non-photosynthetic filamentous bacterium and (ii) at least one agent promoting the synthesis of glycosaminoglycans and (iii) at least one combination of three active agents consisting of an agent increasing the synthesis of collagen, an agent increasing the synthesis of epidermal lipids and an agent promoting the proliferation of keratinocytes.

Examples of these agents for each class are described above.

At least one embodiment comprises at least one Vitreoscilla filiformis extract, a C-β-D-xylopyranoside-2-hydroxy-propane or one of its salts or optical and geometric isomers, a hydrolysed soybean protein, a cinnamic acid or one of its derivatives and a phloroglucinol or one of its derivatives.

Also disclosed herein is the cosmetic use of at least one extract of a non-fruiting non-photosynthetic filamentous bacterium in a composition for reducing the loss of skin density and/or the melting of the lipostructure of the skin and/or avoiding the sagging and/or the hollowing of the volumes of the face, the loss of consistency of the skin and/or its support.

Further disclosed herein is use of at least one extract of a non-fruiting non-photosynthetic filamentous bacterium in a composition for redefining the contours and/or the oval of the face, and/or avoiding the formation of jowls and/or limiting the hollowing of the cheeks and/or of the contour of the eye.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The invention will now be illustrated by the following non-limiting examples. In these examples, the quantities are indicated as a percentage by weight.

### EXAMPLES

### Example 1

### Preparation of a Vitreoscilla filiformis Extract

The Vitreoscilla filiformis strain (ATCC 15551) was cultured according to the method described in international patent application WO-A-94/02158.

This method involved a continuous culture method. The culture was performed at 26° C. for at least 48 hours until a suitable cell concentration corresponding to an optical density at 600 nm greater than or equal to 1.5 was obtained. The strain was subcultured at 2% V/V in fresh medium for about 48 hours until a stable culture was obtained. A 1 liter Erlenmeyer flask containing 200 ml of fresh medium was then inoculated with 4 ml of the preceding culture.

The culture in an Erlenmeyer flask was performed at 26° C. on a culture table agitated at 100 revolutions/minute. The feedstock thus obtained served as inoculum for a 50 liter fermenter. Growth occurred at 26° C., pH 7, 100 revolutions/minute and pO₂≧15%.

After 30 hours of growth, the biomass was transferred to a fermenter with a working volume of 3000 liters in order to be cultured under the same conditions. After 48 hours of growth, the cells were harvested continuously. The biomass was then concentrated about 50 fold by centrifugation. The cells obtained were then frozen as the culture progressed. For the “non-stabilized cell extract,” these cells were used as they were after thawing. For the “stabilized cell extract,” the cells were stabilized by autoclaving at 121° C. for 20 to 40 minutes. The cells were then burst open during sterilization, releasing the cytosol and agglomerating the proteins and the walls. The product obtained was biphasic.

The “supernatant” was obtained by filtering the liquid phase at 0.22 μm in order to remove particles.

The bacterial extract, in cell extract (stabilized or non-stabilized) or supernatant form, can be used as it is (aqueous form) or may be freeze-dried according to conventional techniques (freeze-dried form).

### Example 2

### Demonstration of the Properties of a Vitreoscilla filiformis Extract on Lipogenesis and the Differentiation of Adipocytes

The activity of a Vitreoscilla filiformis cell extract as prepared according to Example 1 on a local adipose development was evaluated. The study was carried out on human adipocytes obtained from the abdominal region. Various parameters representing the adipocyte physiology were analysed:
a) stimulation of lipogenesis by measurement of the incorporation of ¹⁴C-acetate into lipids;
b) stimulation of adipocyte differentiation by measuring the expression of adipocyte differentiation markers: LPL (lipoprotein lipase, involved in the storage of fat;
c) monitoring of the enrichment in lipids by specific staining (AdipoRed™) and imaging;
d) monitoring of the cell size/size distribution by flow cytometry and fluorescent labelling (AdipoRed™).

The Vitreoscilla filiformis extract prepared according to Example 1 was tested at:

10%, 1% and 0.1% for the effect on lipogenesis;

0.25% and 0.05% for the effect on adipocyte differentiation.

Stimulation of Lipogenesis by Measuring the Incorporation of ¹⁴C-acetate into the Lipids

The adipocytes were isolated after an abdominal plastic surgery operation and then incubated in the presence of collagenase and then washed and taken up in test medium. This test medium contained:
product to be tested (Vitreoscilla filiformis extract at various concentrations/theophylline 1 mM/isoproterenol 1 μM), or nothing for the control
bicarbonate 1.87 mg/ml
penicillin/streptomycin 25 IU/ml/25 μg/ml
glutamine 2 mM
MEM without phenol red qs 100% (v/v) supplemented with lipid-free bovine serum albumin at 0.5% (w/v).

The products to be tested and the adipocytes were preincubated at 37° C. for 1 h, and then ¹⁴C-acetate was added at 50 μCi/ml. After 4 h of treatment, the lipids were extracted with methanol/chloroform/water and the incorporated radioactivity was counted by liquid scintillation.

The results are presented in the following table:

| Treatment | | % control | p | % quenching |
|---|---|---|---|---|
| Control | — | 100 | — | 0% |
| Cerulenin | 20 μM | 34 | <0.01 | 0% |
| Isoproterenol | 1 μM | 3400 | <0.01 | 0% |
| Vitreoscilla filiformis extract | 10% | 585 | <0.01 | 66% |
| | 1% | 643 | <0.01 | 0% |
| | 0.1% | 543 | <0.01 | 0% |

Cerulenin (inhibitor of fatty acid synthetase, FAS) tested at 0.02 mM, significantly inhibited the incorporation of acetate into lipids, which validates the test.

The Vitreoscilla filiformis extract significantly stimulated the incorporation of acetate for the 3 concentrations tested, but at the highest concentration, the product interfered with the assay (quenching).

Stimulation of Adipocyte Differentiation by Measuring the Expression of an Adipocyte Differentiation Marker: LPL or Lipoprotein Lipase, Involved in the Storage of Fat

Normal human pre-adipocytes were cultured in PGM growth medium at 37° C. and 5% CO₂ until confluent. At confluence (D0), the cells were placed in differentiation medium (series 1, differentiated cells) or normal growth medium (series 2, undifferentiated cells), containing or not containing (control) the test products or the reference (TNFα, differentiation inhibitor). The cells were then incubated at 37° C. and 5% CO₂ for 4 days.

At the end of the treatment, the supernatants were removed and the cells rinsed in PBS and a volume of 300 μl of Tri-Reagent was added to each well. The expression of the LPL marker was evaluated by RT-Q-PCR on messenger RNAs extracted from the cellular lawns of each treatment. The Light Cycler (Roche Molecular Systems Inc) system was used according to the recommendations of the supplier. The pair of primers which were used make it possible to amplify the following specific fragments:

human β-actin (GenBank No. X00351) as reference;

lipoprotein lipase precursor LPL (GenBank No. M15856).

The incorporation of fluorescence into the amplified DNA was measured continuously during the PCR cycles. A relative expression value was evaluated for the LPL marker, expressed in arbitrary units according to the following formula:
(½^{number of cycles})×10⁶

The results are expressed as % relative expression compared with the control.

| Treatment | | LPL |
|---|---|---|
| Control undifferentiated cells | — | 1 |
| Control differentiated cells | — | 100 |
| TNFα | 25 ng/ml | 111 |
| Vitreoscilla filiformis extract | 0.25% | 17224 |
| | 0.05% | 16947 |

The LPL marker was stimulated with the Vitreoscilla filiformis extract; this indicates a positive effect on lipogenesis, which confirms the effect observed in the preceding test.

Monitoring of the Enrichment with Lipids by Specific Staining (AdipoRed™) and Imaging

At the end of the treatments, the cells were rinsed in PBS and then the intracellular lipids were stained with AdipoRed from BioWhittaker. The stained cells were observed by optical microscopy, with the Spectramax reader.

The results are expressed as % increase relative to the control.

Measurement on differentiated cells

| Treatment | | % control | p |
|---|---|---|---|
| Control (differentiated cells) | — | 100 | — |
| Control undifferentiated cells | — | 65 | <0.01 |
| TNFα | 25 ng/ml | 83 | >0.05 |
| Vitreoscilla filiformis extract | 0.25% | 360 | <0.01 |
| | 0.05% | 150 | <0.01 |

After 4 days of culture in differentiation medium, the adipocytes had an intracellular lipid level increased by a factor of 1.5 relative to the growth medium. TNFα reduced the lipid level as expected.

The Vitreoscilla filiformis extract induced a significant dose-dependent increase in the lipid level in the pre-adipocytes.

The same experiment performed on undifferentiated cells also induced a significant increase in the lipid level in the pre-adipocytes, respectively 564% relative to the control for the concentration of 0.25% of bacterial extract and 196% relative to the control for the concentration of 0.05% of bacterial extract.

Monitoring of the Cell Size/Size Distribution by Flow Cytometry and AdipoRed™ Fluorescent Labelling

At the end of the treatments, the cells were rinsed in PBS and then the intracellular lipids were stained with AdipoRed from BioWhittaker.

At the end of the incubation, the cells were rinsed, trypsinized and then analysed by flow cytometry. The differentiation of the pre-adipocytes manifested itself at the morphological level by an increase in the number of intracytoplasmic lipid vesicles and an increase in the size of the cells.

The results are expressed as % increase relative to the control.

AdipoRed measurement on differentiated cells

| Treatment | | % control | p |
|---|---|---|---|
| Control (differentiated cells) | — | 100 | — |
| Control undifferentiated cells | — | 57 | <0.01 |
| TNFα | 25 ng/ml | 80 | <0.01 |
| Vitreoscilla filiformis extract | 0.25% | 115 | <0.01 |
| | 0.05% | 111 | <0.05 |

The results obtained by this test confirm those obtained above by the measurement with the Spectramax reader.

The same experiment carried out on undifferentiated cells also induced a significant increase in the lipid level in the pre-adipocytes, respectively 131% relative to the control for the concentration of 0.25% of bacterial extract and 124% relative to the control for the concentration of 0.05% of bacterial extract.

All these tests show that the extract of a non-fruiting non-photosynthetic filamentous bacterium had an effect on lipogenesis and the differentiation of the pre-adipocytes into adipocytes. This effect is exploited for the compositions disclosed herein for preventing or reducing age-related hollowing of the face, in particular in the cheeks and the contour of the eye.

### Example 3

### Formulations

1) Anti-face and neck hollowing care cream

| Vitreoscilla filiformis extract according | 0.05% |
|---|---|
| to Example 1 | |
| C-β-D-xylopyranoside-2-hydroxy-propane at 30% | 1.0% as AM |
| by weight of active material (AM) in a 60/40 | |
| water/1,2-propanediol mixture | |
| 1,3,5 Trimethoxybenzene (phloroglucinol) | 0.01% |
| Trans-cinnamic acid | 0.01% |
| Soybean protein extract (Ridulisse ® from SILAB) | 0.5% |
| Oils | 12% |
| Stearyl alcohol | 1.1% |
| Surfactants | 9.0% |
| Emulsifiers | 8.0% |
| Preservatives | 0.75% |
| Perfumes | 0.4% |
| Water | qs 100% |

The anti-face and neck hollowing care cream was applied daily to the skin of the face and the neck, such as the cheeks and the neck in order to avoid hollowing and/or sagging of the skin of the cheeks and the neck.

2) Care of the contour of the eyes

| | |
|---|---|
| Caffeine | 0.2% |
| Vitreoscilla filiformis extract according to Example 1 | 0.1% |
| 1,3,5-Trimethoxybenzene | 0.015% |
| Trans-cinnamic acid | 0.015% |
| Soybean protein extract | 1.0% |
| Silica | 5.0% |
| Oils | 4.0% |
| Fatty alcohols | 3.0% |
| Stearic acid | 3.0% |
| Glycerine | 7.0% |
| Cyclohexasiloxane | 6.0% |
| Surfactants | 3.0% |
| Gelling agents | 1.5% |
| Preservatives | 0.9% |
| Water | qs 100% |

The care product for the contour of the eyes was applied daily to the contour of the eye in order to avoid hollowing of the contour of the eyes.

3) Skin care cream (O/W emulsion)

| | |
|---|---|
| Glycerine | 5% |
| Disodium EDTA | 0.05% |
| Surfactants | 4.0% |
| Preservatives | 0.85% |
| Caprylyl glycol | 0.5% |
| Stearyl alcohol | 1.1% |
| Stearic acid | 3.3% |
| Beeswax | 1% |
| Oils | 21% |
| Vitreoscilla filiformis extract according to Example 1 | 0.025% |
| Soybean protein extract (Ridulisse ® from SILAB) | 0.03% |
| Corn starch | 3% |
| Hydroxypropyl tetrahydropyrantriol | 0.35% |
| Perfumes | 0.4% |
| Acrylamide/sodium acryloyldimethyltaurate copolymer/ | 1.26% |
| isohexadecane/Polysorbate 80 (Simulgel 600) | |
| water | qs 100% |

This care cream was the subject of a cosmetoclinical test described below:

Protocol:

Number of subjects: 38

Inclusion criteria: women under 65 years (average age=58 years) who had been menopausal for at least 3 years, without HRT (Hormonal Replacement Therapy) for at least 6 months, all types of skin, having problems of firmness in the region of the face

Duration of treatment: 4 weeks—twice per day

Method of evaluation: fringe projection

Experimental scheme: randomized

Results:

Under the test conditions, the measurements by fringe projection (or method FOIST) demonstrated a significant increase in the volume of the cheekbones.

What is claimed is:

1. A cosmetic method for reducing the loss of skin density and/or melting of the lipostructure of the skin in the face and/or the neck, and/or for avoiding the sagging and/or hollowing of the volumes of the face, said method comprising applying to the skin of the face and/or the neck a composition comprising, in a physiologically acceptable medium, at least one extract of a non-fruiting non-photosynthetic filamentous bacterium.

2. The cosmetic method according to claim 1, wherein said method is a method for increasing the volume of the cheekbones.

3. The cosmetic method according to claim 1, wherein said skin of the face is chosen from the cheeks, the contours of the eyes and the contours of the face.

4. The cosmetic method according to claim 1, wherein said method is a method for remodelling the face and/or the neck and/or limiting the hollowing of the face of people with mature or very mature skin.

5. The cosmetic method according to claim 1, wherein said method is chosen from a method for remodelling the face and/or the neck and/or limiting the hollowing of the face of menopausal women.

6. The cosmetic method according claim 1, wherein the at least one extract of a non-fruiting non-photosynthetic filamentous bacterium is chosen from a cell extract, the supernatant of said cell extract, and an active fraction of said cell extract.

7. The cosmetic method according claim 6, wherein the at least one extract of a non-fruiting non-photosynthetic filamentous bacterium is a Vitreoscilla filiformis extract.

8. The cosmetic method according to claim 1, wherein the at least one extract of a non-fruiting non-photosynthetic filamentous bacterium is present in the composition in a quantity ranging from 0.001 to 10% by weight of bacterial dry extract relative to the total weight of the composition.

9. The cosmetic method according to claim 8, wherein the at least one extract of a non-fruiting non-photosynthetic filamentous bacterium is present in the composition in a quantity ranging from 0.005 to 2% by weight of bacterial dry extract relative to the total weight of the composition.

10. The cosmetic method according to claim 1, wherein the composition additionally comprises at least one additional agent chosen from: agents promoting the synthesis of dermal and/or epidermal macromolecules, agents stimulating the proliferation of keratinocytes, agents promoting the synthesis of epidermal lipids, agents stimulating cell metabolism and agents promoting microcirculation.

11. The cosmetic method according to claim 10, wherein the composition comprises, in addition to said at least one extract of a non-fruiting non-photosynthetic filamentous bacterium, at least one agent increasing the synthesis of glycosaminoglycans as agent promoting the synthesis of dermal and/or epidermal macromolecules.

12. The cosmetic method according to claim 10, wherein the composition additionally comprises a combination of three active agents comprising
an agent increasing the synthesis of collagen as an agent promoting the synthesis of dermal and/or epidermal macromolecules,
an agent increasing the synthesis of epidermal lipids and
an agent promoting the proliferation of keratinocytes.

13. The cosmetic method according to claim 12, wherein the composition additionally comprises at least one agent stimulating cell metabolism and/or at least one agent promoting microcirculation.

14. The cosmetic method according to claim 13, wherein:
the at least one extract of a non-fruiting non-photosynthetic filamentous bacterium is a Vitreoscilla filiformis extract;
the agent increasing the synthesis of glycosaminoglycans is C-β-D-xylopyranoside-2-hydroxypropane or one of its salts or optical and geometric isomers;
the agent promoting collagen synthesis is a hydrolysed soybean protein;
the agent promoting the synthesis of epidermal macromolecules is a beech Fagus sylvatica bud extract;
the agent increasing the proliferation of keratinocytes is phloroglucinol or one of its derivatives;
the agent promoting the synthesis of epidermal lipids is cinnamic acid or one of its derivatives;
the agent stimulating cell metabolism is a Saccharomyces cerevisiae extract;
the agent promoting skin microcirculation is caffeine.

15. The cosmetic method according to claim 1, wherein the composition additionally comprises at least one screening agent chosen from UVA and UVB screening agents.

16. A cosmetic composition for reducing the melting of the lipostructure of the skin in the face and/or the neck, and/or for avoiding the sagging and/or hollowing of the volumes of the face, said composition comprising, in a physiologically acceptable medium, (i) at least one extract of a non-fruiting non-photosynthetic filamentous bacterium, (ii) at least one C-glycoside derivative.

17. The composition according to claim 16, in which the C-glycoside derivative corresponds to the following formula (I): 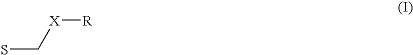
wherein:
R is chosen from:
saturated C₁ to C₂₀ and unsaturated C₂ to C₂₀ linear alkyl radicals, and saturated or unsaturated C₃ to C₂₀ branched or cyclic alkyl radicals; saturated C₁ to C₂₀ and unsaturated C₂ to C₂₀ linear, and saturated or unsaturated C₃ to C₂₀ branched or cyclic, hydrofluoro- or perfluoroalkyl radicals;
wherein the hydrocarbon chain constituting said radicals may be interrupted by 1, 2, 3 or more heteroatoms chosen from:
oxygen,
sulphur,
nitrogen, and
silicon,
and be optionally substituted with at least one radical chosen from:
—OR₄,
—SR₄,
—NR₄R₅,
—COOR₄,
—CONHR₄,
—CN,
halogen atoms,
C₁ to C₆ hydrofluoro- or perfluoroalkyl radicals, and
C₃ to C₈ cycloalkyl radicals,
wherein R₄ and R₅ may be chosen from, independently of each other, hydrogen atoms, saturated C₁ to C₃₀ linear, unsaturated C₂ to C₃₀ linear, and saturated or unsaturated C₃ to C₃₀ branched or cyclic, alkyl, perfluoroalkyl or hydrofluoroalkyl radicals; and C₆ to C₁₀ aryl radicals,
X is a radical chosen from the following groups: 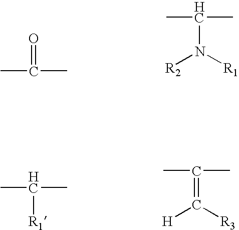
wherein R₁, R₂ and R₃ are chosen from, independently of each other, hydrogen atoms and radicals R, as defined above, and R′₁ is chosen from a hydrogen atom, a —OH group, and a radical R as defined above, and wherein R₁ may also be chosen from C₆ to C₁₀ aryl radicals; and
S is chosen from monosaccharides and polysaccharides containing up to 20 sugar units, in pyranose and/or furanose form and of the L and/or D series, wherein said mono- or polysaccharide may be substituted with a free hydroxyl group, and optionally at least one optionally protected amine functional group, and the S—CH₂—X bond is a bond of a C-anomeric nature, which may be α or β; and its cosmetically acceptable salts, solvates, and its isomers.

18. The composition according to claim 17, wherein S is a monosaccharide chosen from D-glucose, D-xylose, L-fucose, D-galactose, and D-maltose.

19. The composition according to claim 18, wherein S is D-xylose.

20. The composition according to claim 17, wherein X is chosen from —CO—, —CH(OH)—, and —CH(NH₂)—.

21. The composition according to claim 20, wherein X is —CH(OH)—.

22. The composition according to claim 17, wherein R is chosen from linear C₁-C₄, radicals optionally substituted with —OH, —COOH or —COOR″₂, wherein R″₂ is a saturated C₁-C₄ alkyl radical.

23. The composition according to claim 22, wherein R″₂ is an ethyl radical.

24. The composition according to claim 17, wherein the C-glycoside derivative is chosen from C-β-D-xylopyranoside-2-hydroxypropane and C-α-D-xylopyranoside-2-hydroxypropane.

25. The composition according to claim 24, wherein the C-glycoside derivative is C-β-D-xylopyranoside-2-hydroxypropane.

26. A cosmetic composition for reducing the melting of the lipostructure of the skin in the face and/or the neck, and/or for avoiding the sagging and/or hollowing of the volumes of the face, said composition comprising, in a physiologically acceptable medium, (i) at least one extract of a non-fruiting non-photosynthetic filamentous bacterium and (ii) at least one agent promoting the synthesis of glycosaminoglycans and (iii) at least one combination of three active agents comprising an agent increasing the synthesis of collagen, an agent increasing the synthesis of epidermal lipids and an agent promoting the proliferation of keratinocytes.

27. The composition according to claim 26, wherein the composition additionally comprises at least one agent stimulating cell metabolism and/or at least one agent promoting microcirculation.

28. The composition according to claim 27, wherein:
the extract of a non-fruiting non-photosynthetic filamentous bacterium is a Vitreoscilla filiformis extract;
the agent increasing the synthesis of glycosaminoglycans is C-β-D-xylopyranoside-2-hydroxypropane or one of its salts or optical and geometric isomers;
the agent promoting collagen synthesis is a hydrolysed soybean protein;
the agent promoting the synthesis of epidermal macromolecules is a beech Fagus sylvatica bud extract;
the agent increasing the proliferation of keratinocytes is phloroglucinol or one of its derivatives;
the agent promoting the synthesis of epidermal lipids is cinnamic acid or one of its derivatives;
the agent stimulating cell metabolism is a Saccharomyces cerevisiae extract;
the agent promoting skin microcirculation is caffeine.
